SEQUENCE LISTING (6 sequences)

SEQ ID NO: 1 - RNA, 22 nt, Homo sapiens
  scRNA (1)..(22): miRBase Accession, hsa-mir-196a-1(MI0000238) and hsa-mir-196a-2 (MI0000279)
uagguaguuucauguuguuggg

SEQ ID NO: 2 - RNA, 22 nt, Homo sapiens
  scRNA (1)..(22): miRBase Accession hsa-mir-196b MI0001150
uagguaguuuccuguuguuggg

SEQ ID NO: 3 - RNA, 22 nt, Artificial
  primer_bind (1)..(22): single strand RNA, complement to Hsa-miR-196a
cccaacaacaugaaacuaccua

SEQ ID NO: 4 - RNA, 22 nt, Artificial
  primer_bind (1)..(22): single strand RNA, complement to Hsa-miR-196b
cccaacaacaggaaacuaccua

SEQ ID NO: 5 - RNA, 22 nt, Artificial
  primer_bind (1)..(22): single strand RNA, scramble sequence of SEQUENCE NO 3
aacaaauucaacccgucccaaa

SEQ ID NO: 6 - RNA, 22 nt, Artificial
  primer_bind (1)..(22): single strand RNA, scramble sequence of SEQUENCE NO 4
auacaaaucgcacgaaccaacc

(19) United States
(12) Patent Application Publication
(10) Pub. No.: US 2015/0246069 A1
(43) Pub. Date: Sep. 3, 2015

(54) METHOD OF USING SEQUENCE OF ANTAGOMIR FOR DEVELOPING MEDICINE FOR INHIBITING ORAL CANCER CELLS FROM SPREADING

(71) Applicants: Ann-Joy Cheng, Taoyuan County (TW); Ya-Ching Lu, Taoyuan County (TW)

(72) Inventors: Ann-Joy Cheng, Taoyuan County (TW); Ya-Ching Lu, Taoyuan County (TW)

(21) Appl. No.: 14/621,824

(22) Filed: Feb. 13, 2015

Related U.S. Application Data
(63) Continuation-in-part of application No. 13/558,152, filed on Jul. 25, 2012, now abandoned.

(51) Int. Cl. A61K 31/713; A61K 45/06

(52) U.S. Cl. CPC A61K 31/713; A61K 45/06

(57) ABSTRACT

A method of using sequence of antagomiR for developing medicine for inhibiting oral cancer cells from spreading is provided with the steps of combining a sequence of antagomiR with active ingredient to prepare a medicine which is configured to administer to oral cavity grown with oral cancer cells so that both miR-196a and miR-196b of the sequence of antagomiR are configuring of developing medicine for curing oral cancer cells. The miR-196a has a sequence of 5′-UAGGUAGUUUCAUGUUGUUGGG-3′(SEQ.ID.NO.1). The miR-196b has a sequence of 5′-UAGGUAGUUUCCUGUUGUUGGG-3′(SEQ.ID.NO.2).

### CROSS-REFERENCE TO RELATED APPLICATION

This application is a continuation in part of U.S. application Ser. No. 13/558,152, filed Jul. 25, 2012, entitled “METHOD OF USING BOTH MIR-196A AND MIR-196B AS BIOMARKERS FOR DETECTING ORAL CANCER”. The entire teachings of the above application are incorporated herein by reference.

### BACKGROUND OF THE INVENTION

1. Field of the Invention

The invention relates to oral cancer treatments and more particularly to a method of using sequence of antagomiR for developing medicine for inhibiting oral cancer cells from spreading.

2. Description of Related Art

A biomarker is in general a substance used as an indicator of a biological state. It is a characteristic that is objectively measured and evaluated as an indicator of normal biological processes, pathogenic processes, or pharmacologic responses to a therapeutic intervention. Currently, CEA (carcinoembryonic antigen) is used for colorectal cancer indication and PSA (prostate-particular antigen) is used for prostate cancer indication. However, biomarkers for indicating oral cancer are not available as far as the present inventor is aware.

MicroRNA (abbreviated miRNA) is a short ribonucleic acid (RNA) molecule found in eukaryotic cells. A microRNA molecule has very few nucleotides (average of 22) compared with other RNAs.

It is found that miRNA is capable of control target genes based on recent research. Further, different types of cancer have different miRNA molecules based on other recent researches. However, their applications to oral cancer treatment are unknown. Further endeavor is thus desired.

### SUMMARY OF THE INVENTION

One aspect of the invention is to provide a method of developing medicine for inhibiting oral cancer cells from spreading comprising the steps of combining the sequence of antagomiR with active ingredient to prepare a medicine which is configured to administer to oral cavity grown with oral cancer cells so that both miR-196a and miR-196b of the sequence of antagomiR are configuring of developing medicine for curing oral cancer cells wherein the miR-196a has a sequence of 5′-UAGGUAGUUUCAUGUUGUUGGG-3′(SEQ ID NO.1), and the miR-196b has a sequence of 5′-UAGGUAGUUUCCUGUUGUUGGG-3′(SEQ ID NO.2).

Preferably, the oral cancer cells spread includes oral cancer cells migration and invasion.

Preferably, both anti-miR-196a and anti-miR-196b of the sequence of antagomiR are configuring of developing medicine for inhibiting oral cancer cell spreading; and wherein the anti-miR-196a has a sequence of 5′-CCCAACAACAUGAAACUACCUA-3′(SEQ.ID.NO.3) for inhibiting miR-196a, and the anti-miR-196b has a sequence of 5′-CCCAACAACAGGAAACUACCUA-3′(SEQ.ID.NO.4) for inhibiting miR-196b.

Preferably, both anti-miR-196a for inhibiting miR-196a and anti-miR-196a for inhibiting miR-196a show a great reduction of cell migration/invasion ability in OECM1 and SAS cells.

Preferably, both scramble anti-miR-196a and scramble anti-miR-196b are used as a reference group; and wherein the scramble anti-miR-196a has a sequence of 5′-AACAAAUUCAACCCGUCCCAAA-3′(SEQ.ID.NO.5), and the scramble anti-miR-196b has a sequence of 5′-AUACAAAUCGCACGAACCAACC-3′(SEQ.ID.NO.6).

It is therefore one object of the invention to provide a method of developing medicine for inhibiting oral cancer cells from spreading. The invention uses miR-196a and miR-196b as biomarkers for oral cancer detection comprising the steps of analyzing a sample from each of a plurality of human beings in terms of miR-196a and miR-196b wherein the miR-196a has a sequence of SEQ ID NO.1 and miR-196b has a sequence of SEQ ID NO.2; anti-miR-196a has a sequence of SEQ ID NO.3 and anti-miR-196b has a sequence of SEQ ID NO.4; scramble anti-miR-196a has a sequence of SEQ ID NO.5 and scramble anti-miR-196b has a sequence of SEQ ID NO.6; and detecting one of the human beings to have oral cancer if intensity of either miR-196a or miR-196b of the sample belonging to the human being is higher than a predetermined value, wherein the predetermined value of miR-196a is 1.817±0.38 and the predetermined value of miR-196b is 7.683±1.88.

Wherein the method of analyzing a sample from each of a plurality of human beings in terms of miR-196a and miR-196b comprises the sub-steps of:

(a1) sampling blood plasma with each sample having a volume of 200 μl

(a2) extracting nucleic acid by a reagent which does not remove small RNAs;

(a3) adding 700 μl 1QlAzol to each sample for homogeneousness;

(a4) adding 140 μl trichloromethane to each sample to extract RNAs;

(a5) removing about 525 μl of upper pure liquid of the sample to a new tube after using a centrifuge;

(a6) adding 750 μl pure alcohol to the sample to deposit nucleic acid;

(a7) obtaining deposits from the sample by using the reagent;

(a8) draining the sample;

(a9) adding 20 μl water of RNase-free to the sample to extract nucleic acid from the sample;

(a10) for obtaining a constant quantity of nucleic acid, employing a reverse constant polymerase chain reaction reagent;

(a11) employing extracted 3 μl nucleic acid for reverse reaction;

(a12) adding 4 units of reverse reaction enzyme, 10 units of nucleic acid water soluble enzyme inhibition reagent, and 25 mM deoxy-ribonucleoside triphosphate (dNTP) to a reaction chamber having 30 μl volume;

(a13) maintaining the chamber at 37-degree Celsius for about 30 minutes;

(a14) employing a real time polymerase chain reaction (PCR) detection instrument for real time constant quantity PCR;

(a15) combining 8 μl reverse reacted product with 1 μl probe ;

(a16) adding 1 μl water and 10 μl constant quantity PCR reagent to the mixture for reaction; and

(a17) showing results as threshold cycle values in terms of relative presence.

The above and other objects, features and advantages of the invention will become apparent from the following detailed description taken with the accompanying drawings.

### BRIEF DESCRIPTION OF THE DRAWINGS

FIG. 1 is a photograph showing values of 19 miRNA increased and values of 4 miRNA decreased with respect to an oral cancer group and a normal group of cells according to the invention;

FIG. 2A plots mean intensity of 6 oral cancer cell lines versus mean intensity of 5 normal oral keratinocytes;

FIG. 2B shows a table of 6 oral cancer cell lines (C) (mean value), 5 normal oral keratinocytes (mean value), fold (C/N), and P-value in columns;

FIG. 3 depicts oral cancer cells OECM1 and SAS detected by using scramble control (SC) and antagomiR of miR-196a and miR-196b in a wound healing assay; wherein SC means that nucleic acid in the sequence is the same as that in the anti-sequence (e.g., antagomiR, anti-miR-196a and anti-miR-196b) but with random sequence;

FIG. 4 depicts oral cancer cells OECM1 and SAS, and oral cancer cells OECM and SAS by using particular pcDNA (as vector control) and over-expressing plasmid of miR-196a and miR-196b in a wound healing assay;

FIG. 5 depicts oral cancer cells OECM1 and SAS detected by using scramble control (SC) and antagomiR of miR-196a and miR-196b in a cell invasion assay; wherein SC means that nucleic acid in the composition of sequence is the same as that in the anti-sequence (e.g., antagomiR, anti-miR-196a and anti-miR-196b) but with random sequence;

FIG. 6 is a plot similar to FIG. 5 showing the cell invasion ability after whether over-expression of miR-196a and miR196b by plasmid in terms of pcDNA (vector control), miR-196a and miR-196b;

FIG. 7 depicts 52 out of 54 samples being tumor samples in which miR-196a is present in the tumor more than two times as that in the adjacent normal cells in part (a) and 48 out of 54 samples being tumor samples in which miR-196b is present in the tumor more than two times as that in the adjacent normal cells in part (B); and

FIG. 8 plots relative expression (fold) versus samples of blood plasma of a normal person and an oral cancer patient in black spots in part (A), and sensitivity versus specificity for an area under curve (AUC) in part (B), both being in terms of miR-196a; and

FIG. 9 plots relative expression (fold) versus samples of blood plasma of a normal person and an oral cancer patient in black spots in part (A), and sensitivity versus specificity for an area under curve (AUC) in part (B), both being in terms of miR-196b.

### DETAILED DESCRIPTION OF THE INVENTION

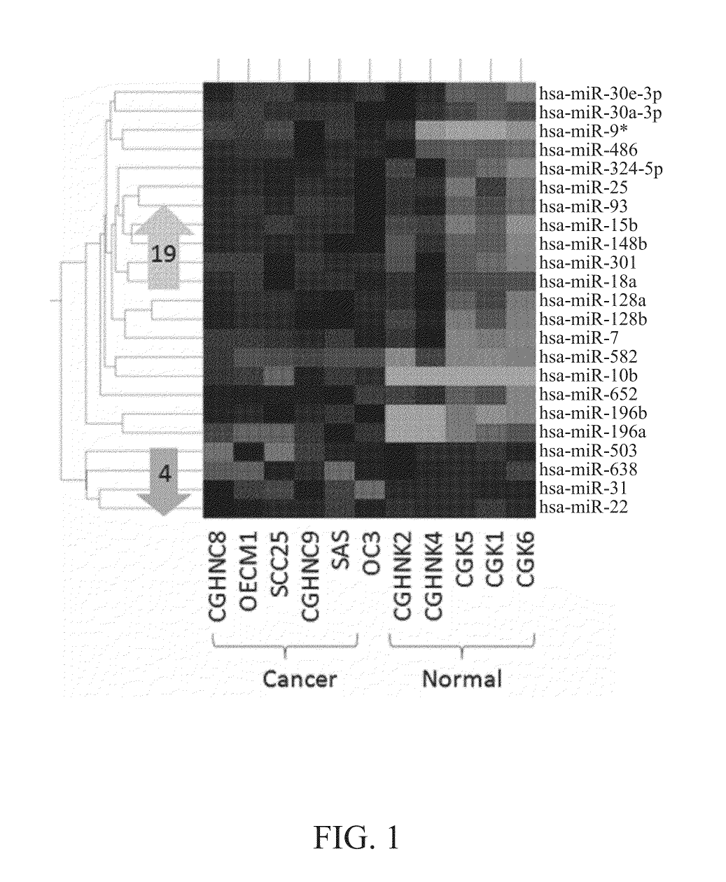

Referring to FIGS. 1 to 9, a method of using sequence of antagomiR for developing medicine for inhibiting oral cancer cells from spreading in accordance with the invention is illustrated below.

It is envisaged by the invention that sequence of antagomiR can be combined with active ingredient to prepare a medicine which can be administered to the oral cavity cancer cell metastasis. Further, both miR-196a and miR-196b of sequence of antagomiR can effectively inhibit oral cancer cells from spreading. Both miR-196a and miR-196b present in the blood plasma, serum, saliva, or tumor of a patient are analyzed. miR-196a has the sequence of 5′-UAGGUAGUUUCAUGUUGUUGGG-3′(SEQ.ID.NO.1), miR-196b has the sequence of 5′-UAGGUAGUUUCCUGUUGUUGGG-3′(SEQ.ID.NO.2), anti-miR-196a has the sequence of 5′-CCCAACAACAUGAAACUACCUA-3′(SEQ.ID.NO.3), anti-miR-196b has the sequence of 5′-CCCAACAACAGGAAACUACCUA-3′(SEQ.ID.NO.4), scramble anti-miR-196a has the sequence of 5′-AACAAAUUCAACCCGUCCCAAA-3′(SEQ.ID.NO.5), and scramble anti-miR-196b has the sequence of 5′-AUACAAAUCGCACGAACCAACC-3′(SEQ.ID.NO.6). Intensity of miR-196a or miR-196b higher than normal value means the patient may have oral cancer. Oral cancer cells spread includes oral cancer cells migration and invasion.

The oral cancer cells spread includes oral cancer cells migration and invasion.

First, nucleic acids of oral cancer cells are analyzed by using miR-196a and miR-196b according to the invention. Intensity of miR-196a and miR-196b in blood plasma of an oral cancer patient is next compared with that of a normal person. It is found that the intensity of miR-196a and miR-196b in blood plasma of the oral cancer patient is much higher than that of the normal person. Thus, it is possible of determining whether a patient has oral cancer in an early stage or not by calculating the intensity of miR-196a and miR-196b of blood plasma of the patient.

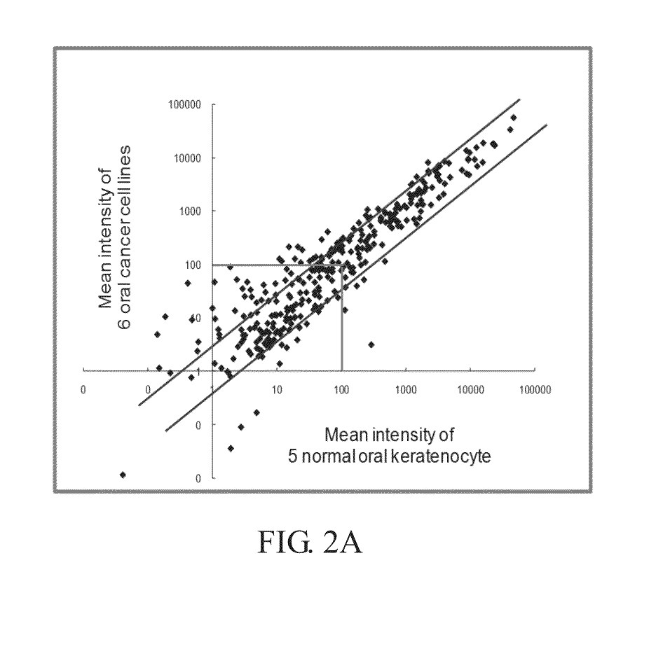

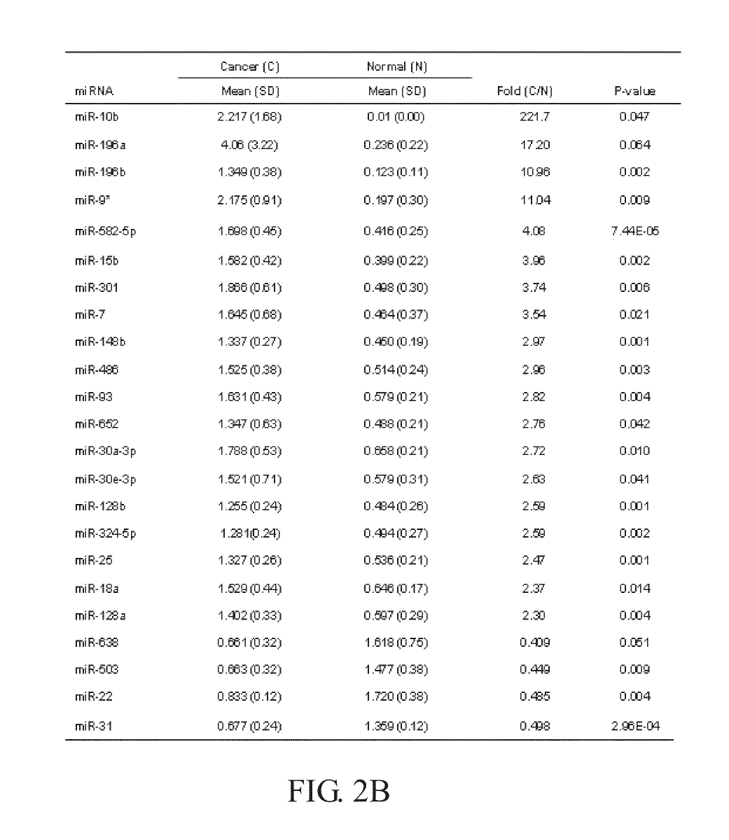

An analysis method developed by Agilent Technology (USA) is employed by the invention to compare nucleic acids in oral cancer cell lines of a patient with that in oral keratinocytes of a normal person. 470 nucleic acids in both 6 oral cancer cell lines of a patient and 5 normal oral keratinocyte are compared with each other and its results are shown in FIG. 2A. In Y-axis, mean intensity of 6 oral cancer cell lines is shown and in X-axis, mean intensity of 5 normal oral keratinocytes is shown.

As shown in FIG. 2B, 23 miRNAs are significantly different from each other in terms of 6 oral cancer cell lines (C) (mean value), 5 normal oral keratinocytes (mean value), fold (C/N), and P-value in columns. Low P-values means substantially the same characteristics of nucleic acids in the same group. Fold (C/N) for miR-196a is 17.20 and that for miR-196b is 10.96. This means that both miR-196a and miR-196b play a great role in oral cancer detection. 470 nucleic acids are detected. Next, clustering analysis of 190 nucleic acids out of the 470 nucleic acids is done after normalization and weak signals removal. This unsupervised hierarchical clustering analysis is shown to divide samples into oral cancer cell lines and normal oral keratinocytes. These two groups are subjected to analysis of variance (ANOVA) with conditions of false discovery rate (FDR) less than 0.1 and more than two times variance. It is indicated that there exists a significant difference among 23 miRNAs with respect to oral cancer cell lines and normal oral keratinocytes. As shown in FIG. 1, values of 19 nucleic acids are increased and values of 4 nucleic acids are decreased with respect to an oral cancer patient and a normal person.

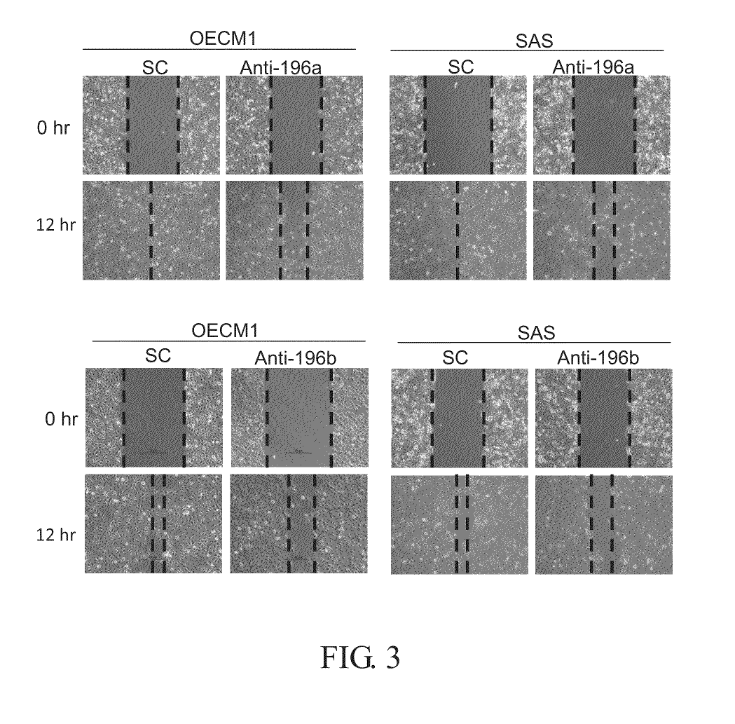

Further, influence of miR-196a and miR-196b for cell migration is analyzed by the invention by conducting a wound healing assay. In detail, two oral cancer cells OECM1 and SAS are detected by using antagomiR, anti-miR-196a of miR-196a, and anti-miR-196b of miR-196b in order to decrease presence level of miR-196a and miR-196b in cells. Therefore, time for cells migrating to gap in the experiment group is greater than that in the control group as shown in FIG. 3. As shown, the oral cancer cells OECM1 and SAS are detected by using scramble control (SC) and antagomiR of miR-196a and miR-196b in the wound healing assay. SC means that nucleic acid in the sequence is the same as that in the anti-sequence (e.g., antagomiR, anti-miR-196a and anti-miR-196b) but with random sequence.

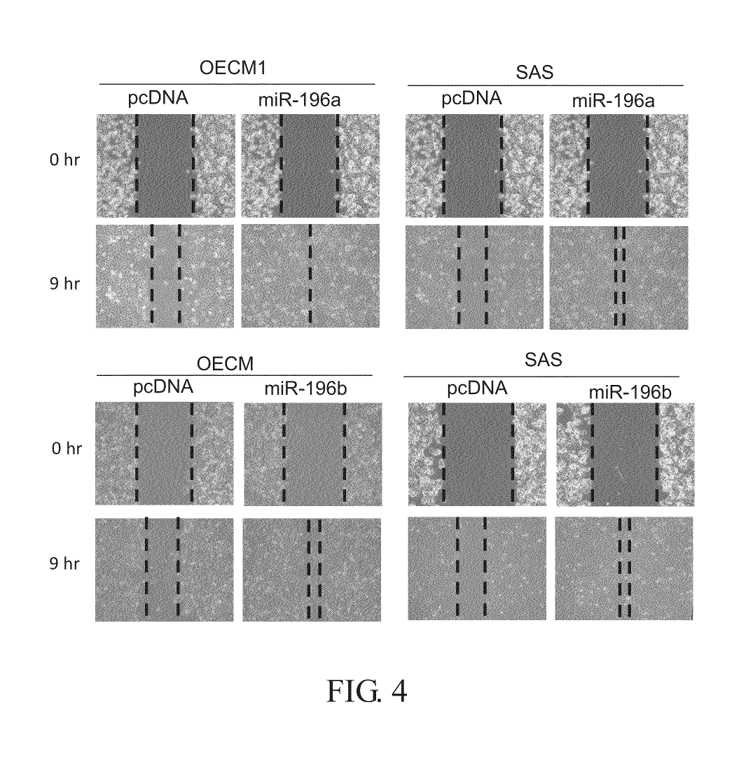

Two cell lines show reduction of miR-196 as or miR-196bs by antagomiR with decreased number in the experiment group do not fill the gap when cells in the control group fill the gap. Moreover, for proving the influence of miR-196a and miR-196b to cells migration, a great number of miR-196as and miR-196bs are present in cells as shown in FIG. 4. As shown, oral cancer cell OECM1 and SAS have transduction of particular pcDNA, miR-196a and miR-196b plasmid. The pcDNA is taken as control group. Subsequently, a wound healing assay is conducted to test cells migration capability. After 9 hours, miR-196a of oral cancer cell OECM1 moves a great distance to fill the gap but pcDNA thereof moves a less distance with the gap not being filled (see top left corner). After 9 hours, miR-196a of oral cancer cell SAS moves a great distance to fill the gap but pcDNA thereof moves a less distance with the gap not being filled (see top right corner). After 9 hours, miR-196b of oral cancer cell OECM moves a great distance to fill the gap but pcDNA thereof moves a less distance with the gap not being filled (see bottom left corner). After 9 hours, miR-196b of oral cancer cell SAS moves a great distance to fill the gap but pcDNA thereof moves a less distance with the gap not being filled (see bottom right corner). In brief, miR-196a or miR-196b can move faster than a cell's particular pcDNA of the control group.

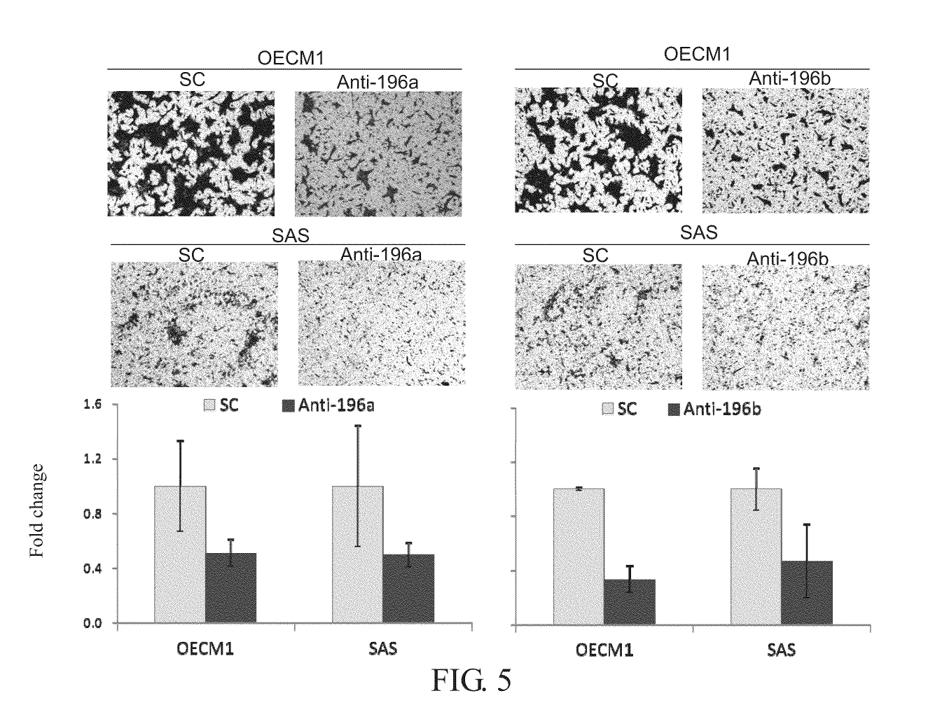

Matrigel invasion assay is employed to determine whether miR-196a or miR-196b can change a cell's invasion capability. Two oral cancer cells OECM1 and SAS are detected by using antagomiR, anti-miR-196a and anti-miR-196b of miR-196a, and miR-196b in order to decrease presence level of miR-196a and miR-196b in cells. In detail, the oral cancer cells OECM1 and SAS are detected by using scramble control (SC) of miR-196a and miR-196b. SC means that nucleic acid in the sequence is the same as that in the anti-sequence (e.g., antagomiR, anti-miR-196a and anti-miR-196b) but with random sequence. This is best shown in FIG. 5. In FIG. 5, anti-196a is anti-miR-196a and anti-196b is anti-miR-196b.

In the two oral cancer cells, antagonized sequence of miR-196a and miR-196b is employed to decrease the presence of miR-196a and miR-196b. Thus, the number of cells passing gel substance of substrate is less as compared with cells in the control group. As compared with cells in the experiment group, the number of OECM1 cells passing gel substance of substrate is decreased to 49% and the number of SAS cells passing gel substance of substrate is decreased to 31% in evaluating the performance of antagonizing miR-196a. Similarly, as compared with cells in the experiment group, the number of OECM1 cells passing gel substance of substrate is decreased to 34% and the number of SAS cells passing gel substance of substrate is decreased to 47% in evaluating the performance of antagonizing miR-196b. In other words, the effect of inhibiting miR-196a by using anti-miR-196a and the effect of inhibiting miR-196b by using anti-miR-196b cause a great reduction of presence levels of both OECM1 cells and SAS cells. The sequence of antagomiR can be used for developing medicine for treating oral cancer by means of the sequence of anti-miR-196a and the sequence of anti-miR-196b. Specifically, both anti-miR-196a for inhibiting miR-196a and anti-miR-196b for inhibiting miR-196b show a great reduction of OECM1 and SAS cells migration/invasion ability. The sequence of antagomiR can be combined with active ingredient to prepare a medicine which can be administered to inhibit the oral cavity cancer cell spreading. In detail, regeant is added to both antagomiR of miR-196a and miR-196b to prepare a medicine which can inhibit both miR-196a and miR-196b levels so as to effectively inhibit oral cancer cells from spreading.

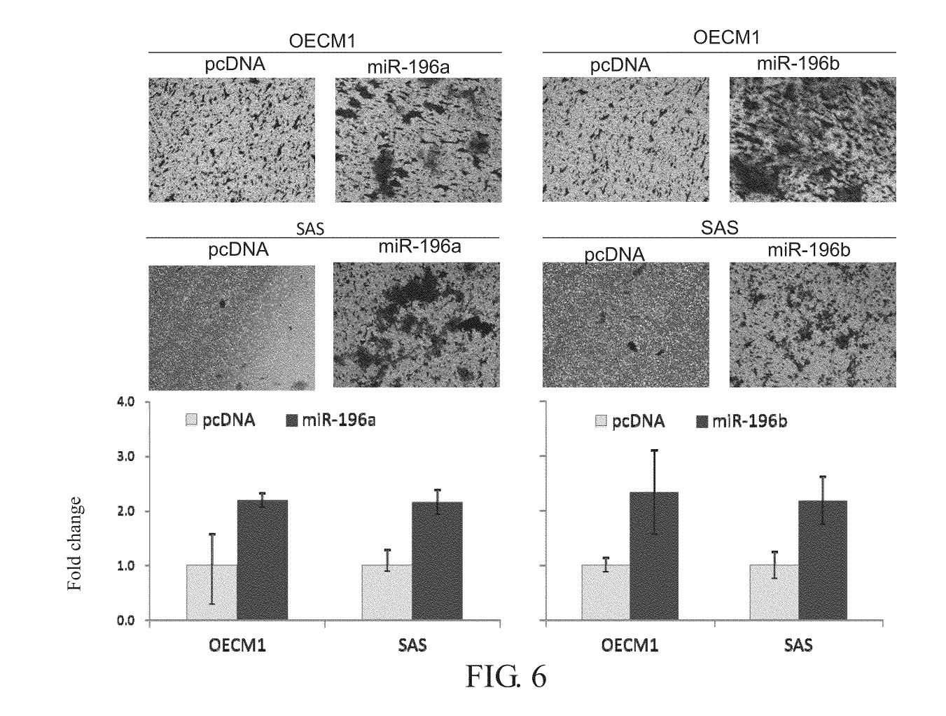

Over-expression of miR-196a and miR-196b in cells in shown in FIG. 6. Over-expression of miR-196a and miR-196b in cells can increase the invasion capability of oral cancer cells. Next, matrigel invasion assay is employed to test the invasion capability of oral cancer cells. Further, cells having particular pcDNA are taken as control group. It is found that the number of high presence miR-196a and miR-196b passing gel substance of substrate to reach the other end of the substrate is about two times as compared with that of cells having particular pcDNA.

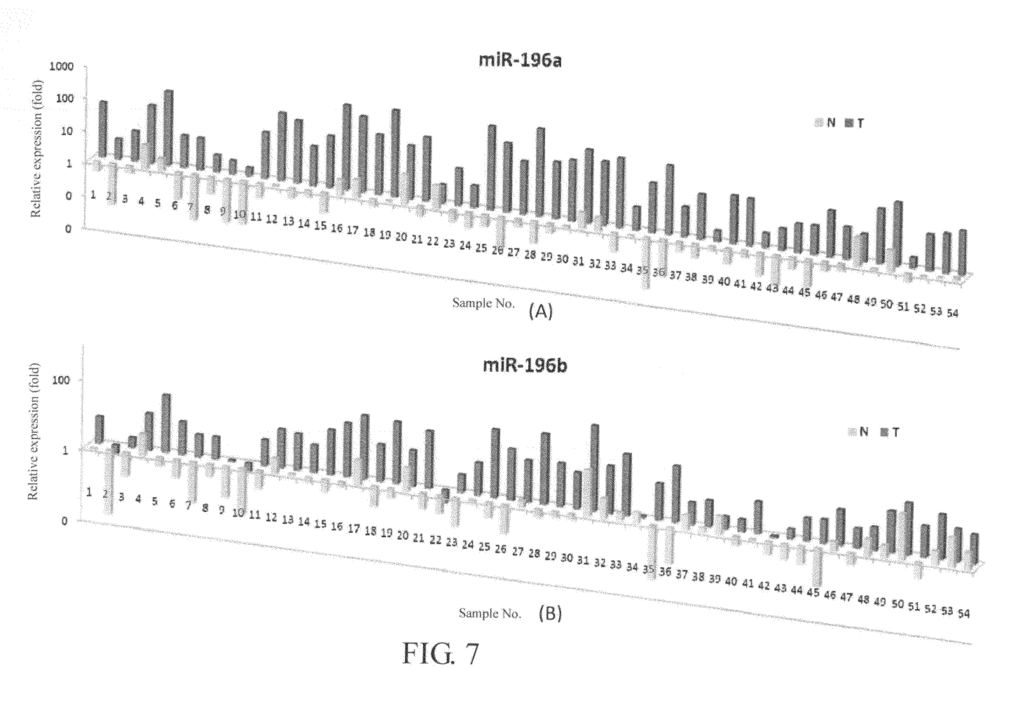

In addition to the oral cancer detection by using miR-196a and miR-196b, the invention also clinically detects presence of miR-196a and miR-196b in a tissue. 54 tumor samples (T) and adjacent normal samples (N) of a patient are detected by the invention. Polymerase chain reaction (PCR) is a scientific technique in molecular biology to amplify a single or a few copies of a piece of DNA across several orders of magnitude, generating thousands to millions of copies of a particular DNA sequence. RT-qPCR is employed by the invention to amplify presence of miR-196a and miR-196b. Further, U6 nucleic acid is taken as internal control of normalization so that results can be expressed in relative presence. In part (A) of FIG. 7, 52 out of 54 samples (i.e., 96.3%) are tumor samples in which miR-196a is present in the tumor more than two times as that in the adjacent normal cells (i.e., p<0.001). In part (B) of FIG. 7, 48 out of 54 samples (i.e., 88.6%) are tumor samples in which miR-196b is present in the tumor more than two times as that in the adjacent normal cells (i.e., p<0.001).

For confirming whether miR-196a and miR-196b can be used clinically, the invention further detects presence of miR-196a and miR-196b in blood plasma of a patient having oral cancer and a normal person respectively. 54 samples of blood plasma of a patient having oral cancer and 33 samples of blood plasma of a normal person are taken and detected. The invention takes the following steps of detecting the presence of miR-196a and miR-196b:

Step 1: Blood plasma is sampled. Samples have a volume of 200 μl. Nucleic acid is extracted from the sample by a reagent which does not remove small RNAs. In detail, miRNeasy® mini kit manufactured by 1QIAgen Inc. in Valencia, Calif. is employed. 700 μl 1QIAzol is added to the sample for homogeneousness. Next, 140 μl trichloromethane (i.e., chloroform) is added to the sample to extract RNAs. About 525 μl of upper pure liquid is removed to a new tube after using a centrifuge. Next, 750 μl pure alcohol is added to the sample to deposit nucleic acid. Further, deposits are obtained by activating the centrifuge to agitate the reagent. The sample is drained from its bottom. Next, 20 μl water of RNase-free is added to the sample to extract nucleic acid from the sample.

Step 2: In a constant quantity method of nucleic acid, reverse constant poly-enzyme chain reaction reagent is employed. miRNA assays kit manufactured by ABI, in Forest City, Calif. is employed as reverse constant poly-enzyme chain reaction reagent. First, extracted 3 μl nucleic acid is employed for reverse reaction. Next, 4 units of reverse reaction enzyme, 10 units of nucleic acid water soluble enzyme inhibition reagent, and 25 mM deoxy-ribonucleoside triphosphate (dNTP) are added to a reaction chamber having 30 μl volume. The chamber is maintained at 37-degree Celsius for about 30 minutes. AMV manufactured by HT Biotech Ltd (UK) and nucleic acid soluble enzyme inhibition reagent manufactured by CalBiochem (CA, USA) are employed. Next, real time PCR detection instrument (e.g., MiniOpticon manufactured by Bio-Rad) is employed for real time constant quantity PCR. 8 μl reverse reacted product and 1 μl probe are mixed. Further, 1 μl water and 10 μl constant quantity PCR reagent are added for reaction. iQ supermix manufactured by Bio-Rad in Hercules, Calif. is employed as constant quantity PCR reagent. Results are shown as Ct value (i.e., threshold cycle) in terms of relative presence.

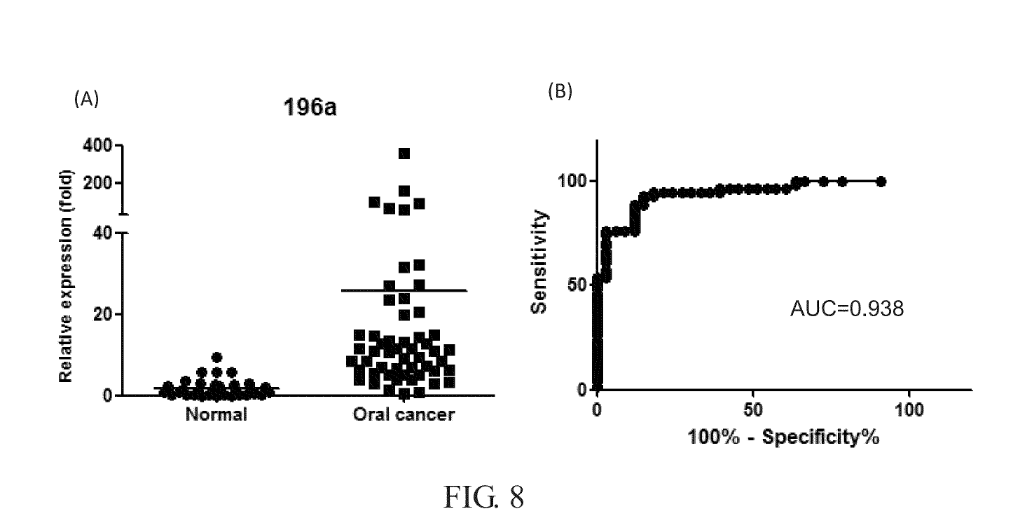

Results show that miR-196a in blood plasma of oral cancer patient increases significantly as compared with that of a normal person. In detail, miR-196a presence in blood plasma of an oral cancer patient is 14.27 with P<0.0001 (i.e., about 14 times of that of a normal person, wherein the value of miR-196a is 1.817±0.38). In part (A) of FIG. 8, samples of blood plasma of a normal person and an oral cancer patient are shown in black spots. Each sample represents a relative expression (fold) of miR-196a measured by RT-qPCT. Black horizontal line represents average of the samples belonging to either a normal person or an oral cancer patient. A chi square (X²) statistic is used to investigate whether distributions of categorical variables differ from one another (i.e., samples of a normal person and that of an oral cancer patient). In part (B) of FIG. 8, a receiver operational curve (ROC) analysis is employed to evaluate the marking capability of miR-196a in both a normal person and an oral cancer patient. Area under curve (AUC) is 0.938 and it means miR-196a can be used as a reliable biomarker for detecting an oral cancer patient out of a great number of normal persons. Logistic regression model is employed to estimate that sensitivity is 92.6% and specificity is 84.6% when miR-196a is used for detecting an oral cancer patient out of a great number of normal persons.

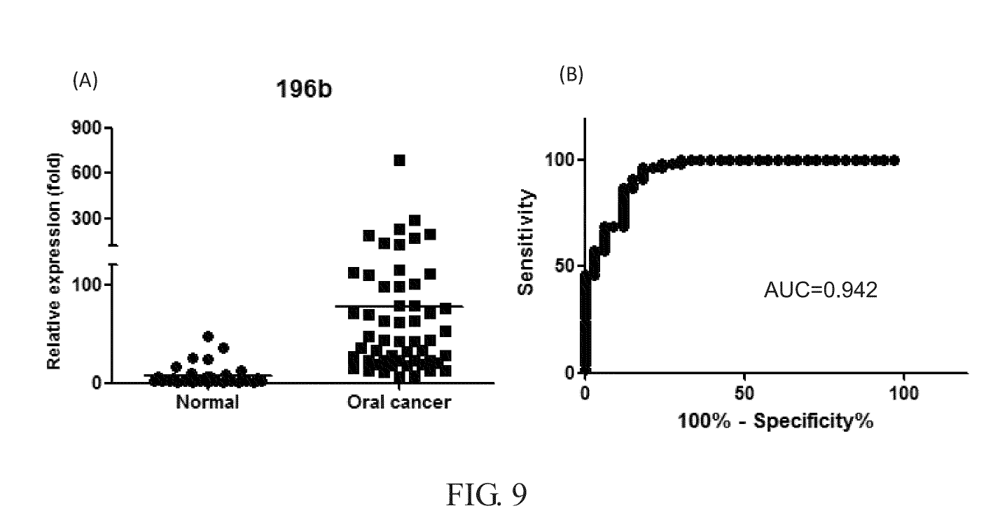

Results further show that miR-196b in blood plasma of oral cancer patient increases significantly as compared with that of a normal person. In detail, miR-196b presence in blood plasma of an oral cancer patient is 10.03 with P<0.0001 (i.e., about 10 times of that of a normal person, wherein the value of miR-196b is 7.683±1.88). In part (A) of FIG. 9, samples of blood plasma of a normal person and an oral cancer patient are shown in black spots. Each sample represents a relative expression (fold) of miR-196b measured by RT-qPCT. Black horizontal line represents average of the samples belonging to either a normal person or an oral cancer patient. A chi square (X²) statistic is used to investigate whether distributions of categorical variables differ from one another (i.e., samples of a normal person and that of an oral cancer patient). In part (B) of FIG. 9, a receiver operational curve (ROC) analysis is employed to evaluate the marking capability of miR-196b in both a normal person and an oral cancer patient. Area under curve (AUC) is 0.942 and it means miR-196b can be used as a reliable biomarker for detecting an oral cancer patient out of a great number of normal persons. Logistic regression model is employed to estimate that sensitivity is 90.7% and specificity is 84.9% when miR-196b is used for detecting an oral cancer patient out of a great number of normal persons.

It is concluded that both miR-196a and miR-196a can be used as a reliable biomarker for detecting an oral cancer patient out of a great number of normal persons.

While the invention has been described in terms of preferred embodiments, those skilled in the art will recognize that the invention can be practiced with modifications within the spirit and scope of the appended claims.

What is claimed is:

1. A method of using sequence of antagomiR for developing medicine for inhibiting oral cancer cells from spreading comprising the steps of:
combining a sequence of antagomiR with active ingredient to prepare a medicine which is configured to administer to oral cavity grown with oral cancer cells so that both miR-196a and miR-196b of the sequence of antagomiR are configuring of developing medicine for curing oral cancer cells wherein the miR-196a has a sequence of 5′-UAGGUAGUUUCAUGUUGUUGGG-3′(SEQ.ID.NO.1), and the miR-196b has a sequence of 5′-UAGGUAGUUUCCUGUUGUUGGG-3′(SEQ.ID.NO.2).

2. The method of claim 1, wherein the oral cancer cells spread includes oral cancer cells growth and migration.

3. The method of claim 1, wherein both anti-miR-196a and anti-miR-196b of the sequence of antagomiR are configuring of developing medicine for curing oral cancer cells; and wherein the anti-miR-196a has a sequence of 5′-CCCAACAACAUGAAACUACCUA-3′(SEQ.ID.NO.3) for inhibiting the miR-196a, and the anti-miR-196b has a sequence of 5′-CCCAACAACAGGAAACUACCUA-3′(SEQ.ID.NO.4) for inhibiting the miR-196b.

4. The method of claim 1, wherein both scramble anti-miR-196a and scramble anti-miR-196b are used as a reference group; and wherein the scramble anti-miR-196a has a sequence of 5′-AACAAAUUCAACCCGUCCCAAA-3′(SEQ.ID.NO.5), and the scramble anti-miR-196b has a sequence of 5′-AUACAAAUCGCACGAACCAACC-3′(SEQ.ID.NO.6).
